(19) United States
(12) Patent Application Publication
(10) Pub. No.: US 2007/0249543 A1
(43) Pub. Date: Oct. 25, 2007

(54) Dissolution of arterial cholesterol plaques by phytochemical emulsifiers

(76) Inventors: Filiberto Zadini, North Hills, CA (US); Giorgio Zadini, Camarillo, CA (US)

Correspondence Address: FILIBERTO ZADINI, 2237 HILLTOP LANE, CAMARILLO, CA, 93012, US

(21) Appl. No.: 11/542,694

(22) Filed: Oct. 4, 2006

Related U.S. Application Data
(60) Provisional application No. 60/793,379, filed on Apr. 19, 2006.

(51) Int. Cl. A61K 31/704

(52) U.S. Cl. 514/26; 514/33

(57) ABSTRACT

A phyto-pharmacological substance, namely a saponin, precursor or derivative, with emulsifying/detergent/surfactant/fat dissolving properties administered into the systemic circulation of a subject via a variety of routes of administration including the topical-dermatological such as a skin patch, the oral-digestive in association with solubility or permeability or stability enhancers, alone or in synergitic combination, the topical-mucous membrane administration such as the sublingual route, the intravenous, subcutaneous, rectal, intramuscular, intradermal, inhalatory in form of inhaled microcrystals, the intrarterial, the saponin substance reaching and dissolving with its emulsifying properties the cholesterol aggregates and in general the lipidic core within the atherosclerotic plaque.

As a result of this phyto-pharmacological direct action upon the atherosclerotic plaque by the phytocompound resulting in the dissolution of the cholesterol aggregates of the plaque, the plaque is no longer vulnerable to rupture and arterial flow is restituted to physiological values present prior to plaque formation. This effect on the lipid core of the plaque by the saponin is expected to reduce and/or eliminate altogether preexisting atherosclerotic lesions and significantly reduce chances of acute and chronic ischemic events.

### RELATED CASES

This application corresponds to Application of Applicants Provisional Patent Application No. 60/793,3379 entitled “Dissolution of Arterial Cholesterol Plaques by Phytochemical Emulsifiers”, filed on Apr. 19, 2006.

### FIELD OF INVENTION

This application relates to phyto-pharmacological compounds useful in the treatment of atherosclerotic plaques aiming at their dissolution and or regression.

### BACKGROUND OF THE INVENTION

Atherosclerosis is a pathological condition responsible of the highest mortality and morbidity in humans.

No known pharmacological compound has unequivocally shown in studies to effectively significantly reduce pre-existing atherosclerotic lesions to the point that clinical benefits would ensue.

Once an atherosclerotic plaque is formed within an artery over the years, such as coronary, cerebral, carotid, iliac, femoral, popliteal arteries, aorta and others, there is little that can be done to reduce its potential for devastating complications or make it disappear altogether and restore arterial anatomical integrity.

There are medications such as statins which act on the serum cholesterol by lowering it significantly. The effect of cholesterol lowering translates into reduced probability of new plaques formation, however, lowering of serum cholesterol does very little to the preexisting plaques.

A few phytocompounds have been reported to be beneficial in the treatment of atherosclerosis. Among them: antioxidants, garlic, flaxseed oil saponins. Antioxidants are contained in many fruits and vegetable and appear to counter the effects of free radicals which are believed to play a major role in atherogenesis. Garlic, which has been reported to lower serum cholesterol. Flaxseed oil, a phytochemical analogous to fish oil which contains omega-3 fatty acids: omega-3 fatty acids appear to lower triglycerids, raise HDL cholesterol “thin” the blood, reduce levels of homoysteine. Saponins which, according to all available studies, are thought to be beneficial in atherosclerosis by lowering the serum cholesterol as a result of their binding to the biliary salts in the gut resulting with reduced emulsification of the ingested fats and consequent hindrance of absorption of ingested fats in the gut. An extensive search in the medical, nutraceutical, phytochemical literature and in the Patent Office has revealed no phytocompound ever being disclosed to act as a lipids emulsifier capable of dissolving pre-existing atherosclerotic plaques by directly acting upon them.

### BRIEF SUMMARY OF THE INVENTION

Applicants have disclosed in their Provisional Patent Application No. 60/739,143, patent application Ser. No. 11/373,943, patent application Ser. No. 11/384,150 a class of physiological emulsifiers, namely biliary compounds, their precursors and/or derivatives as being capable of dissolving the cholesterol aggregates of atherosclerotic plaques.

As disclosed in the above patents applications, Applicants have proved with in vitro experiments that emulsifiers, such as biliary compounds, are capable of dissolving directly the lipidic core of preexisting arterial atherosclerotic plaques.

The concept of an emulsifier acting directly upon the lipidic core of an atherosclerotic plaque and the concept of dissolving the lipidic core via an emulsification process has never been disclosed prior to the filing of the above patents applications.

With the present Provisional patent application Applicants disclose the use of phytochemical compounds, namely saponins, which are natural phytochemical emulsifier/detergent/surfactants, as phyto-pharmacological analogous of biliary compounds, i.e. phyto-pharmacological agents capable of directly dissolving the cholesterol aggregates of atherosclerotic plaques by a process of emulsification similar to the emulsification action of the biliary salts disclosed in the above mentioned patents applications.

Due to the fact that most of the saponins are reportedly poorly absorbed in the gut, Applicants propose alternative convenient routes of administration never disclosed before such as a transdermal route of administration of the compound for instance via a skin patch. Applicants also propose to use intestinal absorption enhancers to favor undigested absorption of the compound.

### OBJECTS OF THE INVENTION

It is an object of the present invention to provide a phyto-pharmacological compound capable of dissolving the lipidic core of preexisting arterial atherosclerotic plaques.

It is an object of the present invention to provide a phyto-pharmacological compound that solubilizes the cholesterol aggregates and other lipid aggregates within the atherosclerotic plaque to such fine particles to enable filtration of such solubilized particles through the fibrous cap of the atherosclerotic plaque into the blood stream in an analog process obtained with biliary salts disclosed in Applicants above mentioned patents applications.

It is an object of the present invention to provide a phyto-pharmacological compound that restores nearly physiological or physiological patency to arterial vessels obstructed by atherosclerotic plaques.

It is an object of the present invention to provide a phyto-pharmacological compound that, by removing the most critical component of an atherosclerotic plaque, i.e. the cholesterol and other lipid content of the plaque, has the ability of contributing to stabilization of the plaque, by minimizing the vulnerability of the plaque to rupture and the consequent ominous thrombus formation.

It is an object of the present invention to provide a phyto-pharmacological compound which has the potential ability of preventing the common complications of atherosclerosis such as acute coronary events and cerebrovascular accidents.

It is an object of the present invention to provide a phyto-pharmacological compound potentially useful in the treatment of peripheral vascular disease, having the potential ability of preventing ischemic limbs disease, and ultimately amputation.

It is an object of the present invention to provide a phyto-pharmacological compound which by restoring patency to the systemic and pulmonary arterial circulation to a near physiological or to a physiological level, has all the prerequisites of likely preventing and curing a number of diseases resulting from inadequate tissue perfusion due to the pathological clogging of the arterial system up to the arterioles. The compound has all the prerequisites of preventing in the long term anoxic damages to the tissues and ultimately probably preventing and in certain cases curing a myriad of pathological conditions originating from, or complicated by, the oxygen tissue deprivation, such as cardiomyopaties, heart failure, senile dementia, vascular complications from diabetes, nephrosclerosis, systemic and pulmonary hypertension, mesenteric ischemias, cerebral atherosclerosis, macular degeneration and probably the cerebral plague of the modern era, Alzheimer disease, likely a result of anoxic chronic insults of various etiology all converging into inadequate cerebral perfusion mainly to the cognition and memory centers.

The concept of exposing the atherosclerotic plaque to the emulsifying activity of saponins is the core of the invention

### FIGURES

FIG. 1 shows a skin patch for systemic administration of the phyto-pharmacological compound.

FIG. 2 shows a device for the administration of the phyto-pharmacological compound, precisely a specially designed intra-arterial catheter for in loco sustained administration of the substance in arteries with atherosclerotic lesions such as coronaries or carotids or popliteal arteries.

FIG. 2A is an enlarged view of the distal segment of the of the device of FIG. 2.

FIG. 2B is an enlarged view of a detail of the device of FIG. 2.

### SPECIFICATIONS

The invention includes a substance or ingredient or active principle or compound or or agent or means, namely a saponin, or a derivative or a precursor of saponins, or a substance structurally and functionally analogous to saponins having the same emulsifying lipid dissolving action as the saponins, being brought to direct contact of the atherosclerotic plaques of arteries of living beings such as human subjects, in a fashion analogous to biliary compounds, as disclosed by Applicants in their Provisional Patent Application No. 60/739,143 and corresponding patent applications Ser. No. 11/373,943 and No. 11/384,150.

As for biliary compounds, saponins require to be made intra-arterially bioavailable for direct contact to atherosclerotic plaques in sufficient quantity to induce lipid depletion of the plaques. As it will be disclosed below, a number of creative routes of administration, more practical than the intravenous route, can be used to achieve intra-arterial bioavailability of saponins.

Besides the intravenous route of administration and other creative routes of administration ultimately leading to intravenous and intra-arterial bioavailability of the saponins, saponins can also be directly applied on atherosclerotic plaques via intra-arterial catheters of the same types of those disclosed by Applicants in patent application Ser. No. 11/384,150, in a topical administration fashion similar or identical to the topical administration disclosed for such intra-arterial catheters.

Saponins are found in many plants, and get their name from the soapwort plant Saponaria, the root of which was used historically as a soap. In Latin sapo means soap. Soaps are detergents which act via emulsification and saponins are glycosides with a distinctive emulsifying characteristic property.

The glycoside of saponins consists of a polycyclic aglycone that is either a steroid, or alkaloid steroid, with the characteristic cyclopentaphenantrene structure, or a polycyclic compound known as triterpenoid, attached via C3 and an ether bond to a carbohydrate side chain. The aglycone is referred to as the sapogenin. While triterpenoid saponins are usually acid, steroid saponins are generally neutral, and are called saraponins. The carbohydrate side chain can be glucose, arabinose, xylose, or glucuronic acid, or other carbohydrate. Glucuronic acid usually combines with the triterpenoid aglucone, while glucose, arabinose and glucose usually combine with the steroid aglucone. Saponins are bitter, however if they have a triterpenoid aglycone they may instead have a licorice taste as glucuronic acid replaces sugar in triterpenoids.

The emulsifying property of a saponin results from the combination of the non polar, hydrophobic, fat-soluble sapogenin, with the polar, hydrophilic, water soluble carbohydrate side chain.

As mentioned above, the steroid saponins, saraponins, such as the diosgenin, the tigogenin, the sarsapogenin, have cyclopentaphenantrene structure in aglycone. Saponins containing nitrogen in aglycone belong also to the steroid saponins group. In the steroid saponins the hydrophilic carbohydrate residue, usually a mono-, di-, tri- or tetrasacharide, is covalently attached to the sapogenin. The sugar moiety, as in most saponins, is attached to the 3-OH of a sapogenin via the 1,2-trans-glycosidic bond. The steroid saponins are the largest class of saponins. To this group belong three classes of compounds: cholestanol, furostanol and spirostanol saponins. Known furostanol steroid saponins are the saponins contained in fresh garlic, such as proto-isoeruboside-B and isoeruboside-B, while aged garlic extract contains also spirostanol steroid saponins. Other examples of spirostanol steroid saponins are the saponin from the underground parts of Ruscus aculeatus, from the rhizomes of Tacca chantrieri, from Solanum hispidum, from the Tubers of Dioscorea polygonoides, from the harvested Tribulus terrestris, from Lilium candidum.

Steroid saponins are also the saponins contained in the root of the Saponaria officinalis, which were used as a soap before the advent of commercially manufactured soap, the saponins of Yucca schidigera, which grows in the arid Mexican desert country of Baja California, used by native American to make soap, the saponins contained in the soap lily, Chlorogalum pomeridianum, which were used as soap by native Americans, the saponins from the soapberry tree.

As for the triterpenoid saponins, an extensive list of triterpenoid saponins from plants is in “Triterpenoids”, by Joseph D. Connolly and Robert A. Hill, Department of Chemistry, Glasgow University, Glasgow, UK G12 8QQ, first published as an Advance Article on the web 16 May 2002.

Among the many triterpenoids saponins listed in the article are the following: fusidane-lanostane group of triterpenoids saponins, such as the cyclopassiflosides saponins, including the cycloglobiseposide saponin, and the cycloartane saponins; the dammarane group of triterpenoid saponins, such as bacopasaponins and jujubogenin saponins; the lupane group of triterpenoid saponins, such as the quadranosides saponins; the oleanane group of triterpenoid saponins, such as the oleanane saponins, including maesapinin saponin, ligatosides saponins, and two saponins, the sandrosaponins and the pedunsaponins, which are found in insect secretions, vulgarsaponin, peduncularisaponin, petersaponins, araliasaponin, assamsaponins, eupteleasaponins, herniariasaponins, jegosaponins, melilotussaponin; the ursane group of triterpenoid saponins, such as randiasaponins, brevicuspisaponins, ursolic acid saponins such as indicasaponins and quadranosides.

Triterpenoid saponins are also the saponins of Quillaja saponaria (soapbark tree), found in arid areas of Chile, where Quillaja bark was used as a shampoo for hundreds of years. Saponins contained in grapes are, as well, triterpenoid saponins.

The above saponins list is by all means not complete. It is only reported to mention instances of the class of saponins found in nature.

The following list includes some of saponins containing plants:

Soapberry and many other members of the family Sapindaceae, including buckeyes Soapwort, conkers/horse chestnuts, Digitalis as digitonin, Grape skin, Olives, Panax as Ginsenoside including Panax Notoginseng rich in saponins content, Gymnostemma Pentaphyllum, Quillaja Saponaria which is member of the Rosaceae family, Soybeans, Yucca, Aloe, Quinoa, Bacopa monnieri, Chlorophytum species, Chlorogalum species soap plants, Tuberous cucurbit species, Medicago sativa, chickpeas Cicer arietinum, seed and foliage, common beans, several rangeland weeds in the US including corn cockle Agrostemma githago, broomweed Gutierrezia sarothrae, Alfombrilla Drymaria arenaroides, Christmas Rose, Helleborus niger, Asparagus fern, Asparagus officinalis, Daisies Bellis perennis, Dioscorea spp, Honeylocust, Fenugreek, Platycodon species, Glycyrrhiza glabra, and many others.

Although saponins are typically found in plants, plants are not the exclusive source of saponins, since some saponins are also found in the animal kingdom, as in most sea cucumbers and starfish, and, as seen earlier, even in insects secretions of Coleoptera, such as Platyphora opima and Desmogramma subtropica.

Difficulties in isolation of homogeneous saponins from natural sources has prompted production of synthetic saponins from laboratory. Examples of synthetic saponins are pamaqueside, tiqueside, synthetic spirostane saponins such as diosgenin and tigogenin glycosides, hecogenin, methyl oleanolate, methyl ursolate, methyl glycyrrhetinate glycosides.

Applicants postulate that, similarly to biliary salts, saponins when placed in contact with an atherosclerotic plaque have the ability of dissolving the cholesterol aggregates and in general the lipidic core of the plaque, ultimately promoting filtration of the emulsified/solubilized cholesterol and lipidic content of the plaque throughout the fibrous cap into the blood stream leaving in situ only a virtual cavity with an intact fibrous cap as the plaque is emptied out of its cholesterol/lipidic content.

Any saponin with detergent/emulsifying activity, either natural or synthetic, alone or in combination, or any precursor or derivative of such a saponin, or any substance structurally and functionally analogous to saponins, alone or in combination, with detergent/emulsifying/surfactant/lipids dissolving properties can be used for the purpose of clearing the arteries from atherosclerotic plaques as long as the saponin being used is able to cross the fibrous cap of the atherosclerotic plaque and access the lipidic core of the plaque.

In order to reach the systemic and pulmonary circulation and act upon the atherosclerotic plaques, saponins can be administered via many routes, including the oral digestive route. The oral digestive route appears however to be largely inefficient with most of the saponins, as only a percentage of the ingested saponins as a rule is absorbed, most of the ingested saponins being, reportedly, inactivated by cholesterin.

Nonetheless Applicants believe that no matter how small the amount of chronically ingested saponins, the percentage of ingested saponin entering the systemic circulation from intestinal absorption is sufficient in the long term to cause beneficial effects not only in the prevention of atherogenesis, but, likely, on preexistent atherosclerotic lesions as a result of direct lipid dissolving properties on the atherosclerotic plaques to be attributed to saponins. This action, postulated by the Applicants to be an attribute of saponins on account of the demonstrated dissolving effect on the atherosclerotic plaques by compounds, such as biliary acids, having the same emulsifying/lipids-dissolving/detergent/surfactant properties as the saponins, is totally distinct from the inhibitory effect on atherogenesis resulting from cholesterol lowering effect demonstrated with oral administration of some saponins.

Chronic exposure of the atherosclerotic plaque to circulating saponins in the blood is expected to result in dissolution of the cholesterol aggregates due to the emulsifying/detergent properties of the saponins.

Saponins direct emulsifying/lipids-dissolving/detergent/surfactant action upon pre-existing atherosclerotic plaques is the novelty introduced by Applicants with this application. This conception is entirely new in the scientific literature. A useful aspect of this new conception is that efforts can be extended to find or synthesize saponins optimized to act directly on pre-existing atherosclerotic plaques to emulsify and dissolve and remove their lipidic core having minimal toxicity.

Applicants report two important studies about beneficial effects of saponins in atherosclerosis “Saponins and phenolic content in plant dietary additives of a traditional subsistence community, the Batemi of Ngorongoro District, Tanzania” T. Johns, R. L. A. Mahunnah, P. Sanaya, L. Chapman and T. Ticktin in the Journal of Ethnoparmacology Volume 66, Issue 1, July 1999, Page 1-10 and “Saponins and Wine”, an unpublished report to the Chemical Society by Andrew L. Waterhouse, Professor, Department of Viticulture & Enology University of California One Shields Ave. Davis, Calif. 95616-8749, http://waterhouse.ucdavis.edu

In the first study the Authors attribute the low incidence of cardiovascular diseases in the Maasai and Batemi populations of East Africa, populations that traditionally consume high levels of dietary fat and cholesterol, to hypocholesteremic plants added to their diet. In the study, plant food additives used by the Batemi of Ngorongoro District, Tanzania, were tabulated, based on interviews with 22 informants, while 17 specimens were collected in the field and analyzed for saponin and phenolic content. A total of 81% of the Batemi additives and 82% of those known to be used by the Maasai contain potentially hypocholesterolemic saponins and/or phenolics.

According to the Authors of the article, herbs and plant parts added to milk and meat-based meals by the Maasai and Batemi populations in East Africa support a role for phenolic antioxidants and hypocholesterolemic agents in the diet, and provide explanation of the low incidence of cardiovascular disease in the Maasai and Batermi populations.

Contrary to the interpretation given by the Authors of the article, Applicants attribute the low incidence of cardiovascular diseases in the Maasai and Batemi populations at least in large part to the lipid dissolving properties of the saponins contained in the herbs and plant parts added to their meals by the Maasai and Batemi populations.

In the study by Dr. Waterhouse “Saponins and Wine” Dr. Waterhouse focuses his attention on the correlation between red wine consumption and decreased mortality rates from coronary artery disease, the so called French Paradox. Dr. Waterhouse found that grapes skin cuticular wax contains a saponin having pentacyclic triterpenoid acids as aglucones, specifically oleanolic acid and its isomer, ursolic acid plus related compounds. The presence of these interesting compounds in wine had never been reported before. According to Dr. Waterhouse “saponins have been shown to have various pharmacological and biological effects. The cholesterol-lowering activity of saponins, and their possible association with decreased incidence of coronary artery disease has been studied for many years. Recent studies have shown, among other effects, links to inflammation control through the COX and 5-LOX pathways. A preliminary study of four red and two white varietal wines from California were analyzed for saponin content. The total saponin concentration of the red wines ranged from 22 to 87 mg/L, and whites from 8 to 12 mg/L. Consequently it appears that Vitis vinifera wines not only contain saponins, but are a significant dietary source, and those found in wine are of particular interest based on their pharmacological properties and possible therapeutic effects. As observed in the French Paradox and other studies, the populations of the Mediterranean and East Asia have very low rates of heart disease mortality, where wine, olive oil, and legumes are important foods in these diets. Thus, the saponins may also be an important contributing factor in explaining the correlation between wine consumption and reduced coronary artery disease mortality.”

The saponins discovered in the wines contain ursolic acid, oleanolic acid, ursolic aldehyde, oleanolic aldehyde, hydroxyhopanone, damarenolic acid, mastidienonic acid isomasticadienonic acid.

As for the case of low incidence of cardiovascular diseases of the Maaasai and Betami populations of the prior study, Applicants attribute the low incidence of coronary artery disease mortality of the populations of the Mediterranean and East Asia regions at least in part to the direct dissolving properties on the athereosclerotic plaques by the circulating phyto saponins being absorbed after ingestion.

Below, Applicants disclose in detail one of the routes which can be used to administer the compounds, a very convenient and easy way, the topical dermatological route by the means of a skin patch.

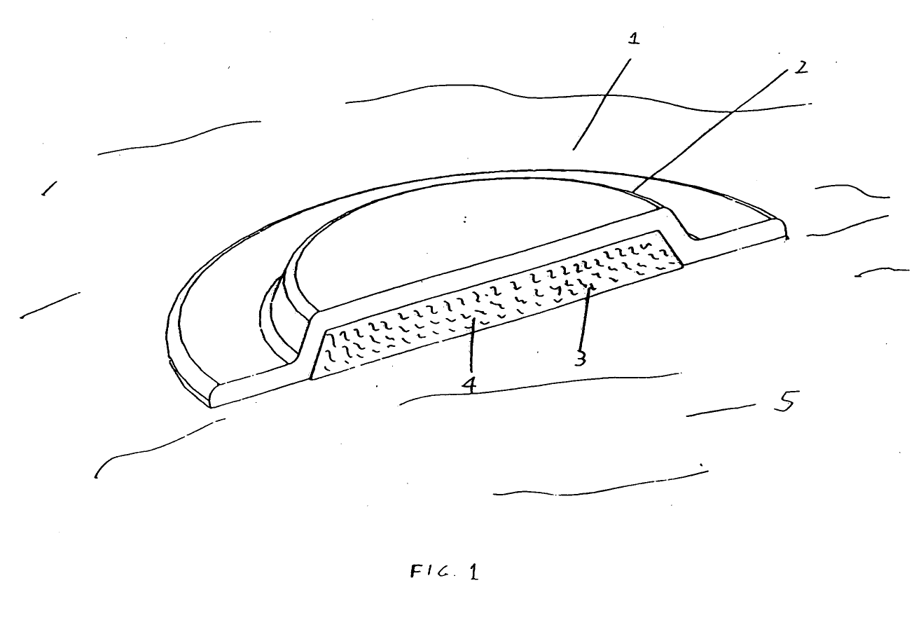

In this embodiment shown in FIG. 1, the ingredient, a saponin, is delivered into the systemic circulation thru the skin in the form of a skin patch impregnated with the saponin compound. The skin patch, generally indicated at 1, shown in FIG. 1, contains a saponin compound designated as 4, either a steroid or triterpenoid saponin, alone or in combination, or any precursor or derivative of saponins, alone or in combination having detergent/emulsifying/surfactant activity.

Skin patch 1, schematically represented in FIG. 1, is composed of two layers, backing/adhesive layer 2 and reservoir layer 3, filled/impregnated with the saponin compound 4 above disclosed.

Backing/adhesive substantially impermeable layer 2 serves the purpose of preventing seeping of saponin compound 4 toward the exterior from patch 1 and serves mainly the purpose of permitting adhesion of patch 1 to skin 5. Reservoir layer 3, composed for instance of interwoven fabric, is impregnated with substance 4, in direct contact with skin 5, serves as reservoir for the delivering of substance 4 thru skin 5 into the systemic circulation.

A skin absorption enhancer such as a permeability enhancer along with ordinary excipents can be added to the saponin in the skin patch to facilitate the penetration and absorption of the saponin thru the skin.

The Percutaneous Absorption Chemical Enhancers which can be added can be classified as: Sulfoxides, Alcohols, Fatty acids, Fatty acid esters, Polyols, Amides Surfactants, Terpene, Alkanones Organic acids, Liposomes, Ethosomes, Cyclodextrins.

Preferably, the Percutaneous Absorption Chemical Enhancers which can be used are: Ethanol, Glyceryl monoethyl ether, Monoglycerides, Isopropylmyristate, Lauryl alcohol, lauric acid, lauryl lactate, lauryl sulfate, Terpinol, Menthol, D-limonene, Beta-cyclodextrin, DMSO acronym for dimethyl sulfoxide, Polysorbates, Fatty acids e.g. oleic, N-methylpyrrolidone, Polyglycosylated glycerides, 1-Dodecylaza cycloheptan-2-one known as Azone®, Cyclopentadecalactone known as CPE-215®, Alkyl-2-(N,N-disubstituted amino)-alkanoate ester, known as NexAct®, 2-(n-nonyl)-1,3-dioxolane known as SEPA®, phenyl piperazine.

The saponin once absorbed in the systemic circulation thru the skin, will act upon the cholesterol aggregates of the atherosclerotic plaque inducing breakdown of the cholesterol aggregates of the arterial plaques, due to the well known physiological emulsifying/surfactant properties of saponins.

In addition to being delivered via skin patch as shown in FIG. 1, the Pharmacological Topical Preparation containing saponins alone or in combination or any precursor or derivative of such saponins alone or in combination, can be delivered into the systemic circulation via semi-solid preparations for application to the skin such as a cream means, ointment means, paste means, gel means, and liquid preparations for application to the skin such as emulsion means, lotion means, colloidal means and the likes.

Percutaneous Absorption Physical enhancers can also be used for transdermal delivery of the above mentioned substances, such as Iontophoresis, Electroporation, Sonophoresis Thermal Poration and in general physically or chemically induced heat, Microneedles, Dermabrasion.

The saponins as disclosed above can be administered via other pharmacological routes of administration:
A) Rectal, for instance in the form of a suppository for direct absorption into the systemic circulation with bypass of the liver and the biliary system
B) Subcutaneous via injection for prompt or slow release delivery of the substance.
C) Intramuscular for prompt or slow release of the substance in a depo form
D) Intravenous
E) Intradermal.
F) Oral mucous membrane, such as sublingual
G) Inhalation in form of inhaled microcrystals or aerosol for pulmonary circulation absorption.
H) Others, such as vaginal or intraperitoneal route

### With regard to the sublingual route, a sweetener can be added to the saponin compounds to improve its palatability due to the bitter taste of certain saponins compounds.

Although the oral ingestion of saponins is associated with unsatisfactory saponins absorption from the intestinal tract, this very practical oral route of administration can be rendered viable and effective with the use of strategies aimed at enhancing enteric absorption of the saponins. To increase intestinal absorption of orally ingested saponins, Applicants propose the use of a number of Enteric Absorption Enhancers: Drug Solubility Enteric Enhancers, Enteric Permeability Enhancers, Drug Stability Enteric Enhancers.

Drug Solubility Enteric Enhancers include: lipid base agents such as liposomes emulsions, microemulsions, self-emulsifying lipid formulations i.e. simple oils, non ionic surfactant and co-surfactants, nanotechnology formulations including cyclodextrins.

Enteric Permeability Enhancers are aimed at rendering the intestinal tract more permeable to saponins. An approach that enhances enteric permeability is bioconjugation i.e. attachments of saponins to non covalent or covalent polymers: these entities also include muco or bioadhesives which stabilize the molecule, maneuver it towards the intestinal lining and increase the residence time at the intestinal wall. Another approach aimed at increasing enteric permeability is nano- and micro-formulations of pharmacologically active ingredients to which formulation the specialized cells lining the intestine such as the Peyer's patch and/or M cells have demonstrated increased permeability: such increased enteric permeability is likely to be exhibited with nano- and micro-formulations of saponins. Another approach aimed at increasing enteric permeability is lipid base formulations: lipid based formulations of saponins are likely to promote their absorption via an increased enteric permeability.

A number of additional factors have shown to increase enteric permeability: sodium glycocholate, sodium taurocholate, sodium deoxycholate, EDTA, sodium salicylate, sodium caprate, diethyl maleate, N-lauryl-beta-D-maltopyranoside, linoleic acid polyoxyethylated, tartaric acid, sodium dodecyl sulpahte, p-t-octyl phenol polyoxyethylene-9.9 known as Triton X-100, Alkylglycosides such as: hexylglucoside, hexylmaltoside, heptylglucoside, octylglucoside, octylmaltoside, nonylglucoside, nonylmaltoside, decylglucoside, decylmaltoside, dodecylmaltoside, tetradecylmaltoside, dodecylglucoside, and tridecylmaltoside, and mucolytic agents such as N-Acetylcysteine.

Enteric Stability Enhancers includes factors capable of promoting controlled release of the active ingredients such as coating, that helps the drug to pass the harsh gastric acidic environment without suffering inactivation, and permits constant or variable pulsatile release, as desired. Among the enteric stability enhancers are also muco- or bio-adhesives which prolong residence of the drug molecule with the intestinal mucosa, with consequent accumulation of the drug in proximity of the enteric lining which in turn promotes passive absorption as a result of increased concentration gradient of the drug. Muco- and bio-adhesives include chitosan, gums, carbomers and derivatives of chitosan, gums and carbomers. Other enteric stability enhancers are inhibitors of pancreatic enzymes and of cellular enzymes that, by preventing or delaying drug degradation in the intestinal environment, ultimately lead to exposure to absorption of a larger percentage of undegraded drug.

The above Enteric Solubility, Permeability and Stability Enhancers can be used alone or in synergistic combination. The above Solubility, Permeability and Stability Enhancers can be integrated for instance in the Liquid Emulsion Drug Delivery System developed by Sigmoid Biotechnologies, called LEDDS. With LEDDS Technology, the rate of uptake and enteric absorption of saponins is expected to be greatly increased. Studies have shown that using of LEDDS minicapsules as a model vehicle for oral delivery of a biopharmacological compound, the formulated drug has entered the blood stream up to four times faster than its conventional equivalent and that a significantly larger percentage of the drug is being absorbed than its conventional equivalent.

At least some of the enteric absorption enhancers discussed above can be used in association with saponins to enhance saponins absorption in routes of administration of saponins involving mucous membranes outside the small intestine, such as in oral mucosal and rectal mucosal absorption, and in the peritoneal absorption.

Aside for the combination of biliary compounds with saponins in order to enhance intestinal absorption of saponins, Applicants advocate the use of combinations of biliary compounds with saponins to synergistically enhance efficacy of lipo-dissolution of atherosclerotic plaques.

As mentioned earlier in this application, saponins can also be directly applied on atherosclerotic plaques via intra-arterial catheters of the same types of those disclosed by Applicants in patent application Ser. No. 11/384,150, in a topical administration fashion similar or identical to the topical administration disclosed for such intra-arterial catheters.

A specialized intra-arterial catheter, such as the catheter described below, allows sustained contact of the saponin in loco, i.e. directly on to the atherosclerotic plaque avoiding dispersion of the saponin in the systemic circulation, for treatment of identified coronary artery or peripheral arteries atherosclerotic lesions. Such a direct application on the plaque allows saponin to act effectively in high concentration upon the plaque virtually without undesired systemic effects

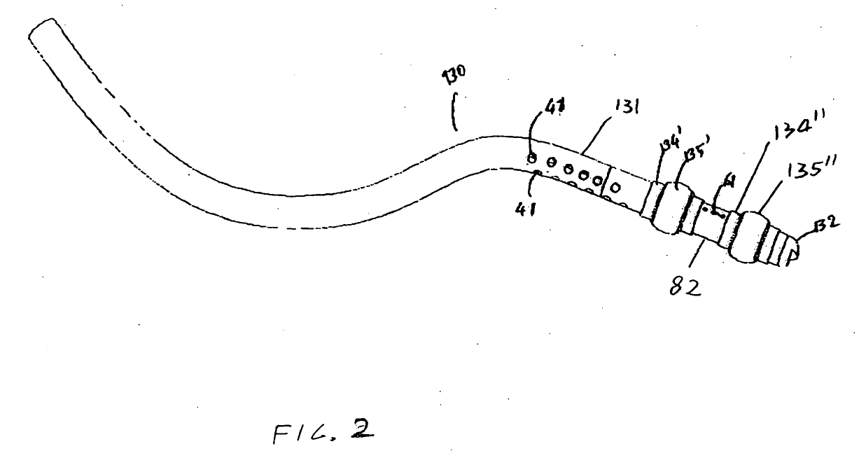

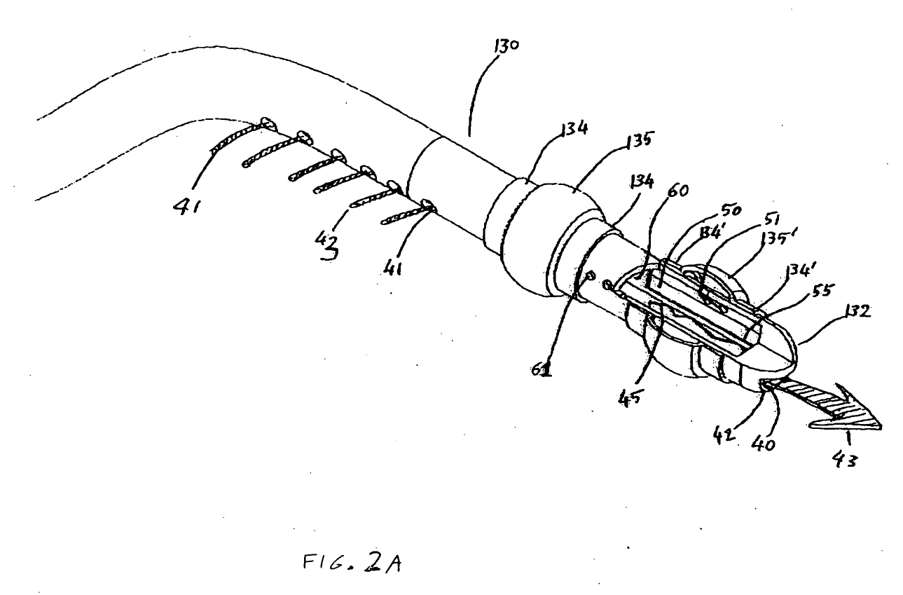

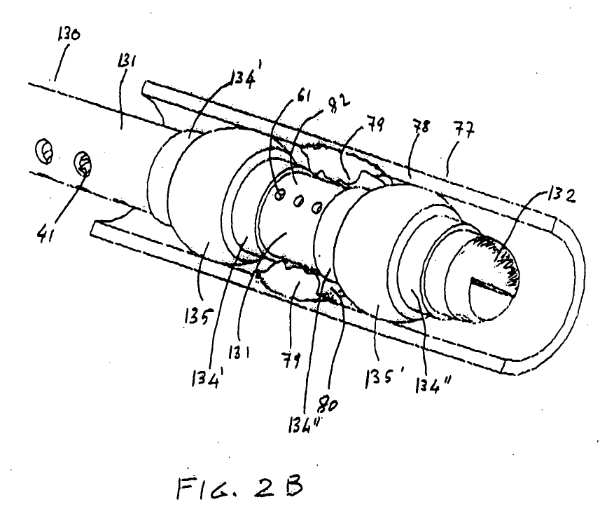

As shown in FIGS. 2, 2A and 2B, catheter 130 is composed of tubular body 131 having distally tip 132, and two generally donut shaped balloons or expandable members, distal balloon, 135″ sealingly connected to tubular body 131 of catheter 130 via sleeves 134″ and a proximal balloon 135′ sealingly connected to tubular body 131 of catheter 130 via sleeve 134′. As better shown in FIG. 2B, balloons 135′ and 135″ are spaced from each other to leave segment 82 of tubular body 131 exposed. As better shown in FIG. 2A, tubular body 131 of catheter 130 has three longitudinal compartments: compartment 40 for passage of blood 43 from inlet openings 41 to outlet openings 42 located at tip 132. This compartment is obliterated proximally to the most proximal inlet opening 41. Septum 45 separates compartment 40 from the other two compartments 50 and 60. Compartment 50 is separated from compartment 60 by septum 55 and is in flow communication with the inside of balloons 135′ and 135″ to allow inflation/deflation of balloons 135′ and 135″. As best shown in FIG. 2B, compartment 60 has openings 61 to allow compound to enter space 80, delimited distally by inflated balloon 135″, proximally by inflated balloons 135′, medially by tubular body 131 of catheter 130 and laterally by the arterial wall 78 of artery 77, which in FIG. 2B is shown longitudinally cross sectioned. Balloons 135′ and 135″ are inflated to a degree to seal space 80 from the remaining segments of artery 77.

In use tip 132 of catheter 130, as better shown in FIG. 2B, is passed in the arterial lumen beyond atherosclerotic plaque 79 of arterial wall 78 of artery 77 so as to align exposed segment 82 of tubular body 131 with atherosclerotic plaque 79. The phytochemical compound object of the present invention is introduced into compartment 60 at the proximal end of catheter 130, to fill space 80 in suitable concentration and for an extended period of time to exert its full dissolving effect on atherosclerotic plaque 79 of arterial wall 78 of artery 77. The compound can then drained from the proximal end of compartment 60, and after balloon deflation, the catheter is removed from the artery. The above description of catheter 130 is purely illustrative of a method for direct application of the phytochemical compound on the lesioned arteries where the compound can be applied at high concentration on the arterial wall and sealed off from the arterial blood which is bypassed within the artery to avoid dispersion of the compound in the blood stream and to maximize the effect of the compound on the atherosclerotic plaques. Other known types of catheters having two discrete balloons or a dog bone shaped balloon can be used for drug delivery applications, to seal off the precise area that requires treatment. Additional intracoronary or generally intra-arterial drug delivery catheters can be used for such purpose, with different designs, such as the Dispatch by SciMed, which is multichamber autoperfusion balloon catheter, or the Channel Balloon Catheter by Boston Scientific, a local drug-delivery catheter that has the dual capability of high-pressure lesion dilation and low-pressure drug infusion.

Saponins can be used alone via the routes disclosed above or in combination with the following compounds:
1) Statins, with the purpose of clearing the blood from the expected transitory cholesterol increase resulting from the lipidic dissolution of the atherosclerotic plaques induced by the emulsifying action of the saponin, to impede new plaque formation being achieved by the action of the statins which effectively lower serum cholesterol.
2) EDTA with the purpose of removing the calcium deposits frequently present within the atherosclerotic plaques.
3) Lipase to add a lipolytic activity to the emulsifying activity of the saponin possibly in a synergistic fashion.
4) Collagenase for the purpose of enhancing the permeability the fibrous cap of the atherosclerotic plaque and accelerating and/or facilitating and/or enhancing the penetration of saponin into the plaque.
5) Hematoporfyrins which have shown to selectively accumulate within atherosclerotic plaques in a study once administered intravenously. The complex saponins with hematoporfyrins would enhance in loco delivery of the complex into the atherosclerotic plaque by selective localization and accumulation of the complex in the atherosclerotic plaques.

What we claim is:

1. A treatment for atherosclerotic plaques of living beings, having a lipidic core mainly consisting of cholesterol aggregates, and a fibrous cap covering the lipidic core, comprising:
a saponin, wherein said saponin is brought to direct contact of said atherosclerotic plaque to cause a process of emulsification of the lipidic core of the plaque.

2. The saponin of claim 1 wherein said saponin being brought to contact of said atherosclerotic plaque is in a quantity sufficient to cause regression of said atherosclerotic plaque via said process of emulsification.

3. The saponin of claim 1, wherein said saponin is introduced into the blood stream of the systemic circulation of a living being in quantity sufficient to cause regression of said atherosclerotic plaque via said process of emulsification.

4. The saponin of claim 3, wherein said saponin is introduced into the blood stream of the systemic circulation via transdermal delivery.

5. The saponin of claim 4, wherein said transdermal delivery of said saponin comprises a skin patch.

6. The saponin of claim 4, wherein said transdermal delivery of said saponin comprises a preparation for application to the skin.

7. The saponin of claim 4, wherein said transdermal delivery of said saponin is enhanced via percutaneous absorption chemical enhancers.

8. The saponin of claim 4, wherein said transdermal delivery of said saponin is enhanced via percutaneous absorption physical enhancers.

9. The saponin of claim 3, wherein said saponin is introduced into the blood stream of the systemic circulation via a rectal route of administration for direct absorption into the systemic circulation with bypass of the liver and the biliary system.

10. The saponin of claim 3, wherein said saponin is introduced into the blood stream of the systemic circulation via a route of administration comprising a subcutaneous injection.

11. The saponin of claim 3, wherein said saponin is introduced into the blood stream of the systemic circulation via a route of administration comprising an intramuscular injection.

12. The saponin of claim 3, wherein said saponin is introduced into the blood stream of the systemic circulation via a route of administration comprising an intravenous injection.

13. The saponin of claim 3, wherein said saponin is introduced into the blood stream of the systemic circulation via a route of administration comprising an intradermal injection.

14. The saponin of claim 3, wherein said saponin is introduced into the blood stream of the systemic circulation via an oral mucosa route of administration.

15. The saponin of claim 3, wherein said saponin is introduced into the blood stream of the systemic circulation via a route of administration comprising pulmonary absorption.

16. The saponin of claim 3, wherein said saponin is introduced into the blood stream of the systemic circulation via a route of administration comprising generally mucosal absorption.

17. The saponin of claim 3, wherein said saponin is introduced into the blood stream of the systemic circulation via a route of administration comprising peritoneal absorption.

18. The saponin of claim 3, wherein said saponin is introduced into the blood stream of the systemic circulation via an oral route of administration in combination with an enteric absorption enhancer.

19. The saponin of claim 1, wherein said saponin is introduced into the human body via a catheter for in situ delivery of said saponin for sustained contact of said saponin directly on to the atherosclerotic plaque of an artery while the saponin is being sealed off from blood bypassed within the artery.
